(19) United States
(12) Patent Application Publication
(10) Pub. No.: US 2014/0142570 A1
(43) Pub. Date: May 22, 2014

(54) HIGH-FREQUENCY APPLICATION DEVICE FOR VASCULAR USE, IN PARTICULAR FOR APPLICATION OF HIGH-FREQUENCY ENERGY TO THE RENAL ARTERIAL WALL

(71) Applicant: BIOTRONIK AG, Buelach (CH)

(72) Inventors: Frank Bakczewitz, Rostock (DE); Nicolas Degen, Beringen (CH); Eugen Hofmann, Zuerich (CH)

(73) Assignee: BIOTRONIK AG, Buelach (CH)

(21) Appl. No.: 14/076,884

(22) Filed: Nov. 11, 2013

Related U.S. Application Data
(60) Provisional application No. 61/728,251, filed on Nov. 20, 2012.

(51) Int. Cl. A61B 18/14

(52) U.S. Cl. CPC A61B 18/14
USPC 606/41

(57) ABSTRACT

A high-frequency application device for vascular use, in particular for application of high-frequency (HF) energy to the renal arterial wall, including: a catheter (1) with a lumen (4) passing through it in the longitudinal direction; a self-expanding stent-like support (6) guided in the lumen (4); and an HF applicator (9) arranged on the support (6) for delivering HF energy to bodily tissue, wherein the HF applicator (9), as a multipole arrangement, has a plurality of HF contact elements (14) distributed axially and peripherally over the support (6), which are insulated from the support (6) and are connectable to an HF source (10) for simultaneous or sequential delivery of HF energy to different positions of the bodily tissue.

### CROSS REFERENCE TO RELATED APPLICATIONS

This application claims benefit of priority to U.S. patent application Ser. No. 61/728,251 filed Nov. 20, 2012; the content of which is herein incorporated by reference in its entirety.

### TECHNICAL FIELD

The invention relates to a high-frequency application device for vascular use, in particular for application of high-frequency (HF) energy to the renal arterial wall.

### BACKGROUND

Such a high-frequency application device is known as a result of prior public use and comprises a catheter with a lumen passing through it in the longitudinal direction, a self-expanding stent-like support guided in the lumen, and an HF applicator arranged on the support for delivering HF energy to bodily tissue.

This known HF application device has just a single HF applicator as a single-pole ablation electrode. If, for therapeutic purposes, HF energies are to be applied in a bodily cavity or bodily vessel at a number of positions offset from one another, for example as is the case with RSD (renal sympathetic denervation) therapy, this single ablation electrode is associated with the disadvantage that the procedure has to be repeated a number of times per vessel at different positions in order to ensure the success of the therapy. Up to six repetitions per vessel are normal. Since HF energy has to be applied to each individual ablation point for up to two minutes, the intervention as a whole is very time-consuming. With the single-pole method, the ablation points cannot be positioned very precisely, since, after each application of HF energy, the catheter has to be manually displaced axially and also in a circumferential direction over a specific path.

Other approaches, known as a result of prior public use, for solving the above problem are based on the use of a balloon catheter. However, this has the disadvantage that a curved artery is directed in a straight line upon balloon dilation, which is associated with the risk of rupture of the vessel. In addition, balloons of different size have to be used for different vessel diameters.

Lastly, braided stent designs have the disadvantage of demonstrating a very significant change in length during their expansion. This likewise hinders accurate positioning of the ablation points. In addition, the pressure of the electrodes against the arterial wall can only be adjusted with difficulty.

Proceeding from the aforementioned disadvantages of the prior art, the object of the invention is to create a high-frequency application device for vascular use, in which HF energy can be delivered simultaneously to a number of locations.

### SUMMARY

This object is achieved, in accordance with the characterizing part of claim 1, by a high-frequency application device, in which the HF applicator, as a multipole arrangement, has a plurality of HF contact elements distributed axially and peripherally over the support. These are insulated from the support and are connectable to an HF source for simultaneous or sequential delivery of HF energy to different positions of the bodily tissue.

The HF application device according to the invention thus makes it possible to deliver HF energy simultaneously to a number of positions, for example to the inner vessel wall of a renal artery. The ablation process for each artery thus takes place over just a short period of time, as would be necessary in the prior art for the application of HF energy to a single ablation point. The duration of the painful ablation procedure is thus reduced many times over. The HF contact elements can be freed in the artery due to the arrangement on a self-expanding stent-like support (for example made of shape-memory metal such as Nitinol). Once the HF energy has been delivered, the support is retracted back into the catheter, or the catheter is slid back over the support, and can thus be removed from the artery. The system is thus not only self-expanding, but can also be repositioned.

Preferred developments of the high-frequency application device according to the invention are characterized in the dependent claims. The HF contact elements may thus each have a freed contact zone and at least one connecting web forming their mechanical connection to the support. As a result of this embodiment, the individual functions of the mechanical holding of the contact elements and the HF energy delivery are assigned to different components, namely the contact zone and the connecting web. These components can therefore each be tailored optimally to their task.

Furthermore, the high-frequency application device can be designed in the region of each of the contact zones such that the position of these zones can be varied between a passive position resting against the unexpanded support or embedded therein and an active position protruding radially beyond the outer contour of the expanded support. The therapeutically effective contact zones thus protrude radially beyond the contour of the edge of the stent-like support structure and ensure sufficient contact with the vessel wall, even in winding passages of arteries. The contact zones are arranged at defined axial and peripheral distances from one another in this instance, whereby successful therapy is to be achieved in a reliably predictable manner.

Different embodiments of the contact zones are conceivable. The contact zone may thus be designed as a closed therapeutic contact surface having a flat, paddle-like form. These contact zones are manufactured relatively easily together with the support structure in terms of the production process, for example by being cut from a tubular material.

Alternatively, the contact zone may form a mechanical holder, on which a separate therapeutic contact surface is arranged. For example, this can be designed as an HF electrode head, which is arranged in a receptacle of the holder.

The HF electrode head is then advantageously decoupled galvanically from the holder by local insulation, and is ideally simultaneously coupled thermally, as effectively as possible, to the metal support structure. To this end, the HF electrode can be glued to, or in, the metal support structure, for example by means of thermally conductive yet electrically insulating adhesives. It is also possible to cast the HF electrode integrally with the metal support structure using a polymer.

The HF electrode head can be supplied with energy in the conventional manner via individual wires, although energy supply via a printed circuit board, which sits on the support and on which the HF electrode head is assembled, is advantageous.

Individual annular surfaces for forming the therapeutic contact surfaces of the respective contact zones may also be provided on the support for the purpose of galvanic decoupling.

A further alternative for the insulation of the contact zone lies in an insulation layer, for example a thin plastics layer, as a coating over the entire support or over part of the support.

The contact zones can be reliably positioned in a variable manner relative to the support as a result of a further possible length-flexible design of the connecting webs of the contact elements between the respective contact zone and the support, said webs in particular extending in a meandering manner. Reliable contacting of the application device against the vessel wall can thus be assisted.

Since, with HF applications in vessels, point-specific temperature monitoring of the location to which energy is applied is advantageous, one or more temperature sensors may be provided in, or on, the HF contact elements. The temperature in the vicinity of the therapeutic contact surface can thus be checked in an ongoing manner.

Further preferred embodiments characterize basic structures, known per se, for the stent-like self-expanding support. This can also be designed in the manner of what is known as a slotted tube stent, also referred to hereinafter as a “slotted tube” for short. This stent structure is cut from a tube, for example by means of a laser beam, and therefore forms a closed design in contrast to a braided design. In addition, this stent structure does not demonstrate a change in length that is relevant in practice during the expansion process, whereby very accurate positioning of the ablation points in the peripheral and axial direction is made possible. Significant advantages compared to the single-pole ablation apparatus known from the prior art, which is based on a braided design of the stent, are thus achieved. The rate of success when subjecting the sympathetic nerves in the renal artery for example to sclerotherapy increases significantly compared to this braided design stent as well as single-pole application.

Further advantages of the slotted tube stent compared to the braided design lie in the smaller profile expansion, since the points of intersection of the wire braid present in the braided design are omitted. Furthermore, insulation can be implemented more easily by a polymer coating after shape-setting. Insulated wires in the braided design do not allow any temperature treatment for shape-setting in the stent structure. Furthermore, as a support structure, slotted tube stents have a lower torsional rigidity than the extremely torsionally rigid braided design structures. The radial force of the HF contact elements, which for example are formed as paddle-like attachments, can also be set in an improved manner.

Greater versatility of the stent design can be cited as one advantage compared to balloon technology. Curved vessels are not straightened during treatment (dilated balloon), thus reducing the risk of damage to the vessel. In addition, the blood flow through the meshes of the slotted tube stent is practically uninterrupted.

An alternative for the design of the support is a type of stent graft or the combination of a plurality of a number of self-expanding annular segments, which are assembled in succession on a bearing shaft displaceable in the catheter. Both versions have advantages similar to those of the slotted tube stents detailed above.

In accordance with a further development of all embodiments, variants and alternatives of the described high-frequency application device, the HF contact elements are manufactured from a material having good X-ray contrast.

### DESCRIPTION OF DRAWINGS

Further features, details and advantages of the invention will become clear from the following description of exemplary embodiments based on the drawings, in which:

FIG. 1 shows a schematic overview of a high-frequency application device,

FIG. 2 shows a schematic plan view of an HF applicator having a slotted tube design,

FIG. 3 shows the distal end of the HF application device according to FIG. 1 with the HF applicator in the active position,

FIG. 4 shows a schematic plan view of an HF applicator with a stent graft design,

FIG. 5 a schematic view of the distal end of a high-frequency application device with an HF applicator formed of a plurality of self-expanding annular segments,

FIG. 6 shows a plan view of an HF contact element in a first embodiment,

FIG. 7 shows a plan view of an HF contact element in a second embodiment,

FIG. 8 shows an axial section of the HF contact element along the line of section A-A according to FIG. 6,

FIGS. 9 and 10 show an axial section of the HF contact element similar to FIG. 8 in two further different embodiments,

FIGS. 11 and 12 show plan views of an HF applicator with a support having a slotted tube design in the collapsed and expanded state,

FIG. 13 shows a schematic perspective view of the HF applicator according to FIGS. 11 and 12 in the expanded state,

FIGS. 14 and 15 show plan views of an HF applicator in a further embodiment in the collapsed and expanded state, and

FIG. 16 shows a plan view of an HF applicator formed as an individual segment.

### DETAILED DESCRIPTION

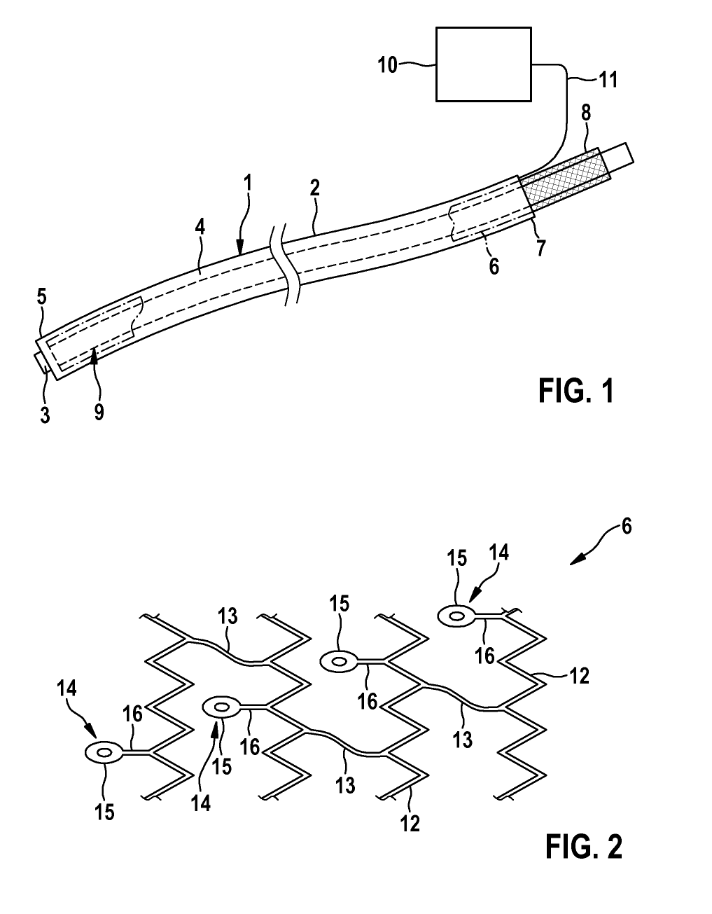

As can be seen from FIG. 1, a high-frequency application device for vascular use has a catheter 1 formed as an elongate tube with an outer shaft 2 and an inner shaft 3 arranged therein. An annular lumen 4 is formed between these shafts and passes through the catheter 1 in the longitudinal direction.

A support 6 that is stent-like at least at the distal end 5 is arranged in this lumen 4 and is to be actuated at its proximal end 7 by a schematically indicated actuation mimic 8 in a manner that is yet to be described in greater detail. At the distal end 5 of the support 6, an HF applicator denoted on the whole by 9 is provided, for example to apply HF energy for complete or partial transection or traumatization of sympathetic nerves at the renal artery for lasting therapy of chronic hypertension. This HF energy is not generally used to completely transect or destroy the nerve physiologically, but to make it incapable of function as a result of processes induced by the HF energy.

FIG. 1 shows a purely schematic illustration of an HF source 10 for supplying energy to the HF applicator 9, said HF source being connected to the HF applicator 9 via a suitable line 11.

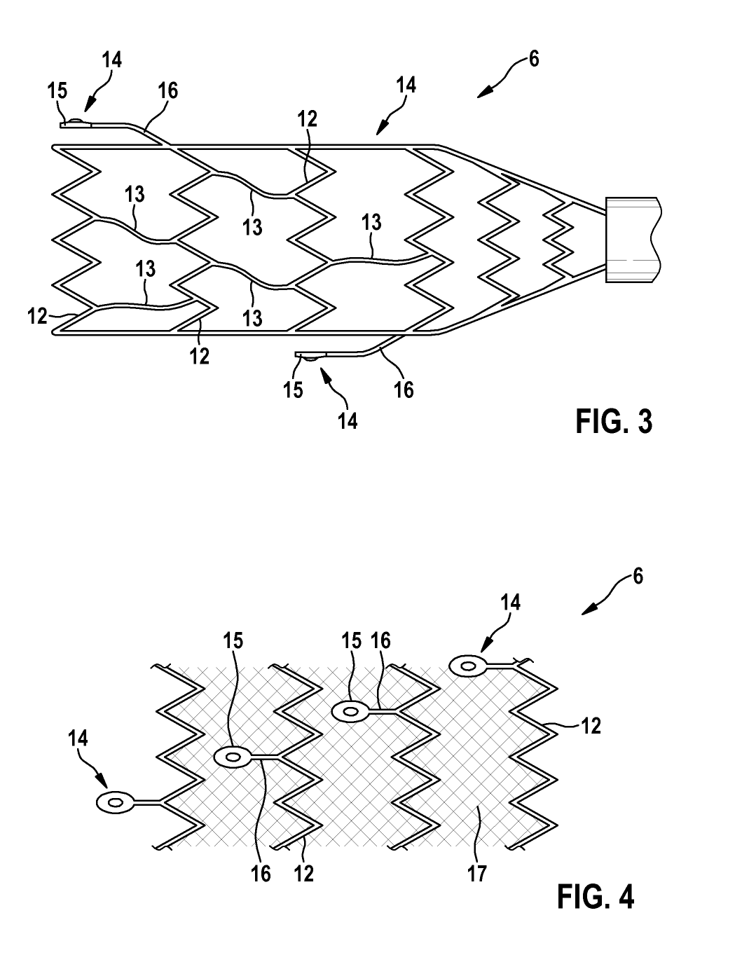

A first embodiment for the HF applicator 9 is illustrated in FIGS. 2 and 3. The support 6 is formed in this case in the manner of a slotted tube stent, which forms a type of net structure from main meander struts 12 and longitudinal bridge struts 13. HF contact elements 14 are distributed over the support 6 at various meander points of the main meander struts 12 and are each connectable as an electrode to the HF source 10 for the delivery of HF energy at different positions of the bodily tissue.

With use of the high-frequency application device, the catheter 1 is advanced via its distal end 5 to the corresponding position within the body, together with the support 6 retracted into its lumen 4, as indicated in FIG. 1. Once in this position, the outer shaft of the catheter 1 is withdrawn, so that the self-expanding support 6 expands when it exits from the lumen 4, as shown in FIG. 3.

The HF contact elements 14 are each formed in this case by a contact zone 15, freed from surrounding material of the support 6 by corresponding cutouts, at a connecting web 16 carrying said contact zone for mechanical connection thereof to the support 6. As can be seen in FIG. 3, the contact zones 15 of the HF contact elements 14 are displaced radially outwardly as a result of the expansion of the support 6, such that a reliable contact between the contact zones 15 and the bodily tissue, for example of the renal artery, surrounding the HF applicator 9 is ensured. In this state, the contact zones 15 can then be supplied by the HF source 10 with corresponding HF energy, and corresponding ablations can be carried out at the contact points for therapeutic purposes.

FIG. 4 illustrates an alternative embodiment for the support 6, which in this case is designed in the manner of a stent graft. This again has main meander struts 12, which are interconnected in the longitudinal direction by a flexible woven fabric 17 however. Similarly to the embodiment according to FIGS. 2 and 3, HF contact elements 14 again sit on the main meander struts 12.

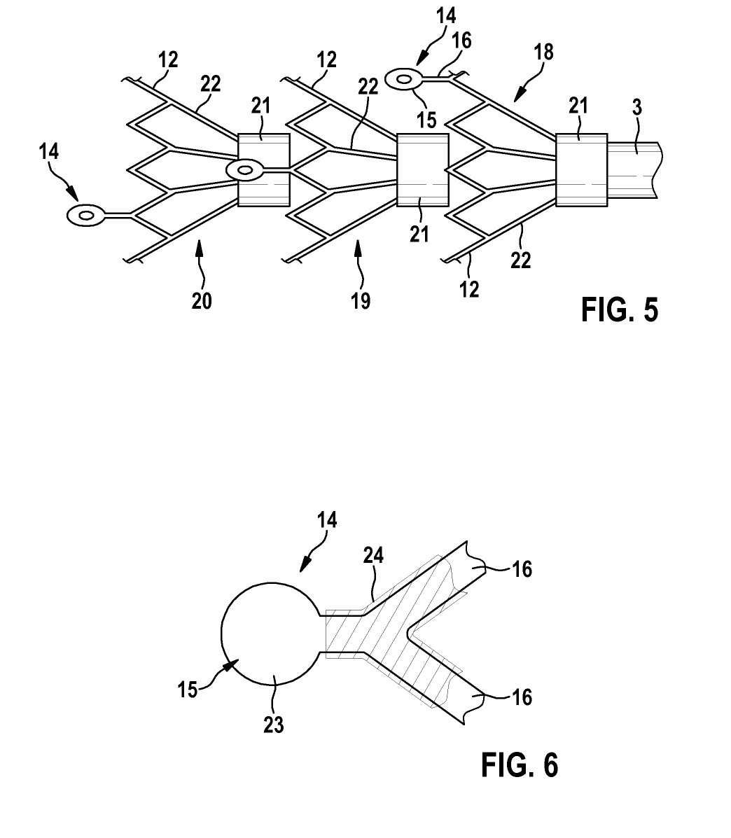

In the embodiment shown in FIG. 5, the support 6 is formed of a plurality of self-expanding annular segments 18, 19, 20, which are each fastened on the inner shaft 3 of the catheter 1 via sleeves 21. Similarly to the embodiments according to FIGS. 2 and 4, each annular segment again has main meander struts 12 with HF contact elements 14 fitted thereon. The main meander struts 12 are in this case connected to the sleeves 21 via longitudinal coupling struts 22. As can be seen clearly on the basis of FIG. 5, the annular segments 18, 19, 20 are folded together in an umbrella-like manner when the inner shaft 3 is retracted into the outer shaft 2 of the catheter 1, whereby the stent-like annular segment structure contracts. Inversely, the annular segments 18, 19, 20 expand when the outer shaft 2 is withdrawn over the inner shaft 3, whereby the HF contact elements 14 again contact the inner wall of the vessel.

Different embodiments of the HF contact elements 14 are to be explained on the basis of FIGS. 6 to 10. FIG. 6 thus shows an HF contact element 14, of which the contact zone 15 is formed as a closed therapeutic contact surface 23 having a flat, paddle-like form. This is decoupled galvanically from the connecting webs 16, and thus from the rest of the support 6, in a suitable manner, for example by a thin plastics coating 24.

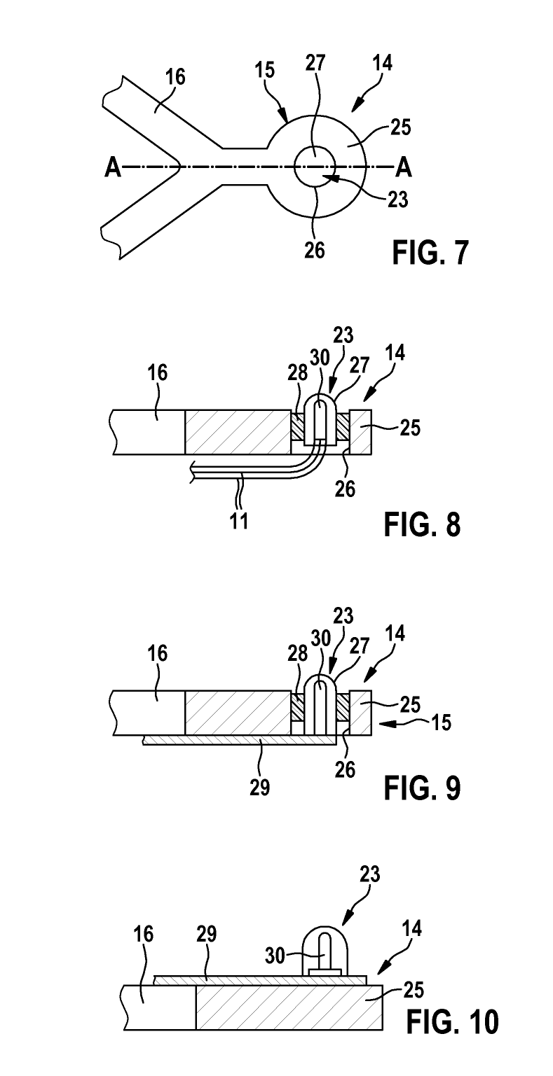

The variant illustrated in FIGS. 7 and 8 shows an HF contact element 14 having a contact zone 15, which forms an annular mechanical holder 25 in the form of an aperture 26. An HF electrode head 27 is housed in this aperture 26 as a therapeutic contact surface 23, which is insulated galvanically in the aperture 26 via a suitable ring insulator 28. The electrode head 27 itself is supplied with HF energy via the above-mentioned lines 11, as also shown in FIG. 8.

In the embodiment illustrated in FIG. 9, the HF electrode head 27 likewise sits in a galvanically decoupled manner via the ring insulator 27 in the aperture 26 of the mechanical holder 25, which is formed by the contact zone 15, but a printed circuit board 29 is in this case provided beneath the contact zone 15, the HF electrode head 27 being assembled on said printed circuit board and being connected accordingly to the HF source 10 via strip conductors (not illustrated in greater detail).

In the embodiment according to FIG. 10, the HF electrode head 27 is likewise assembled on a printed circuit board 29, wherein this sits on the mechanical holder 25 however, such that the aperture 26 can be omitted. The HF electrode head 27 is again supplied with energy via strip conductors on the printed circuit board 29.

With the HF electrode heads 27 shown in FIGS. 7 to 10, a temperature sensor 30 is integrated and is used to measure the temperature in the direct vicinity of the ablation location. The application of HF energy to the bodily tissue can thus be controlled in a particularly reliable manner.

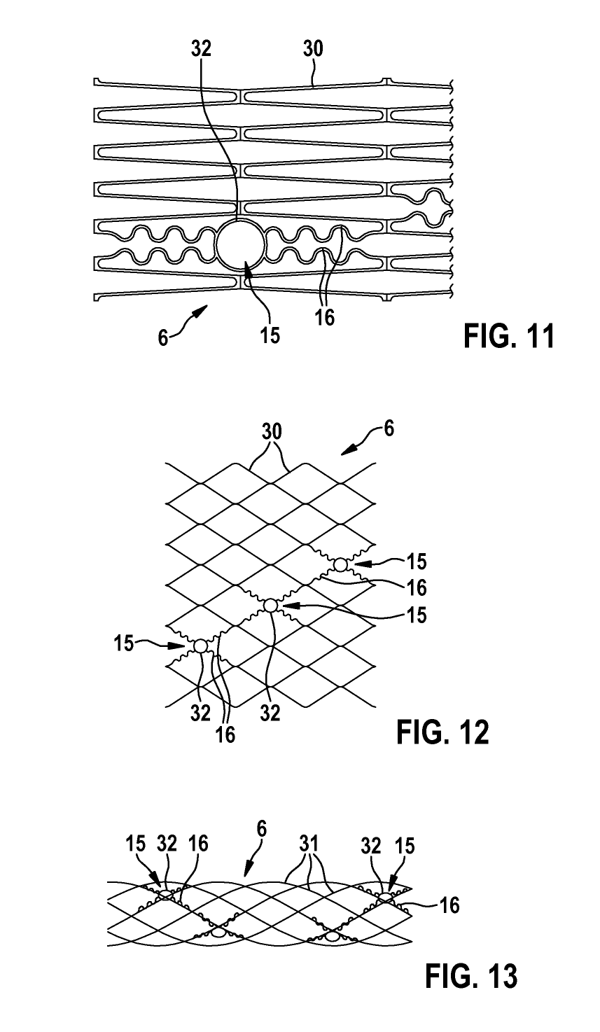

FIGS. 11, 12 and 13 show a support 6 based on a slotted tube design with lattice struts 31 arranged in a diamond-shaped manner, wherein annular surfaces 32 are formed as contact zones 15 at different points of this structure and are connected to the structure of the support 6 via meandering connecting webs 16.

As is clear from FIGS. 12 and 13, the meandering connecting webs 16 compensate for the expansion movement of the lattice struts 31 and ensure that the annular surfaces 32 remain far outwards in the radial direction and protrude radially beyond the contour of the support 6.

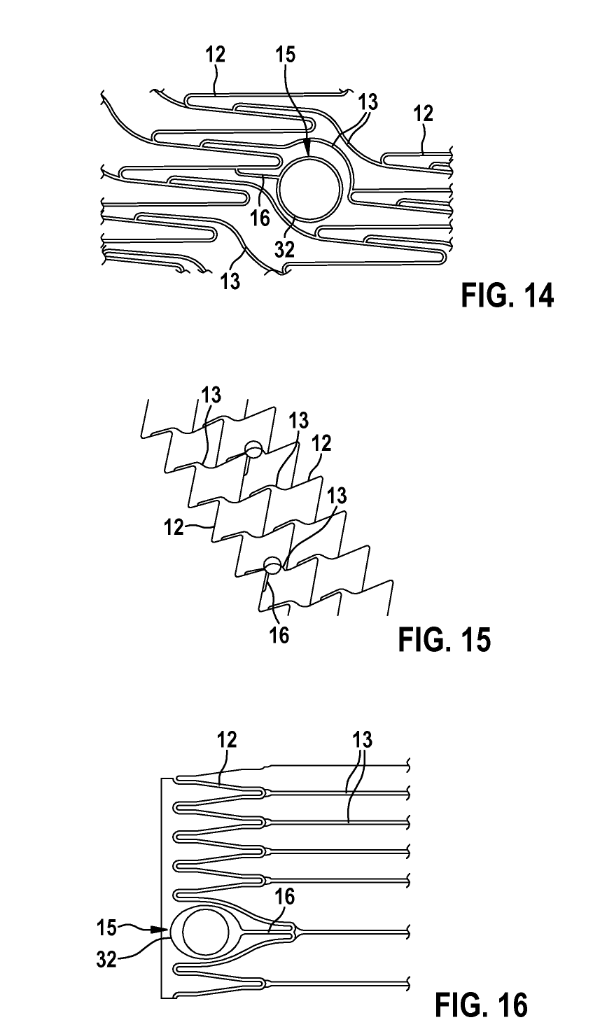

A further example of a support design with main meander struts 12, curved longitudinal bridge struts 13 and a contact zone 15, designed as an annular surface 32, of the HF contact elements 14 is shown in FIGS. 14 and 15. The contact zones 15 are in this case connected to the main meander struts 12 via a single, narrow connecting web 16. As can be seen from FIG. 15, the annular surfaces 32, which, in the contracted position, are embedded into the structure between two curved bridge struts 13, slide outwardly beyond the bridge struts 13 during the expansion process, whereby the contact with the surrounding tissue is again ensured.

The basic designs of the support 6 shown in FIGS. 11 to 13 and 14 and 15 are known in principle as a “closed-cell” slotted tube design (closed cell design), apart from the additions provided in accordance with the invention.

Lastly, an individual segment having main meander struts 12 and longitudinally extending bridge struts 13 is illustrated in FIG. 16, wherein a contact zone 15 formed as an annular surface 32 is again connected between two meander curves to the main meander struts 12 via a connecting web 16.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A high-frequency application device for vascular use, in particular for application of high-frequency (HF) energy to the renal arterial wall, said device comprising:
a catheter with a lumen passing through it in a longitudinal direction,
a self-expanding stent-like support guided in the lumen, and
an HF applicator arranged on the support for delivering HF energy to bodily tissue,
characterized in that
the HF applicator, as a multipole arrangement, has a plurality of HF contact elements distributed axially and peripherally over the support, which are insulated from the support and are connectable to an HF source for simultaneous or sequential delivery of HF energy to different positions of the bodily tissue.

2. The high-frequency application device as claimed in claim 1, characterized in that the HF contact elements each have a freed contact zone and at least one connecting web for their mechanical connection to the support.

3. The high-frequency application device as claimed in claim 2, characterized in that the position of the contact zone of the respective HF contact element can be varied between a passive position resting against the unexpanded support or embedded therein and an active position protruding radially beyond the outer contour of the expanded support.

4. The high-frequency application device as claimed in claim 2, characterized in that the contact zone is formed as a closed therapeutic contact surface having a flat, paddle-like form.

5. The high-frequency application device as claimed in claim 2, characterized in that the contact zone forms a mechanical holder, on which a therapeutic contact surface is arranged.

6. The high-frequency application device as claimed in claim 5, characterized in that the therapeutic contact surface is formed as an HF electrode head, which is arranged in a receptacle of the holder.

7. The high-frequency application device as claimed in claim 6, characterized in that the HF electrode head is galvanically decoupled from the holder by local insulation.

8. The high-frequency application device as claimed in claim 6, characterized in that the HF electrode head, for energy supply, is assembled on a printed circuit board, which sits on the holder.

9. The high-frequency application device as claimed at least in claim 2, characterized in that the therapeutic contact surface of the contact zone is formed as an annular surface.

10. The high-frequency application device as claimed in claim 1, characterized in that the contact zones are insulated by an insulation layer on the support.

11. The high-frequency application device as claimed in claim 2, characterized in that the connecting web is flexible in length, in particular as a result of a meandering progression.

12. The high-frequency application device as claimed in claim 1, characterized in that one or more temperature sensors are arranged in, or on, the HF contact elements.

13. The high-frequency application device as claimed in claim 1, characterized in that the support is formed in the manner of a slotted tube stent.

14. The high-frequency application device as claimed in claim 1, characterized in that the support is formed in the manner of a stent graft.

15. The high-frequency application device as claimed in claim 1, characterized in that the support is composed of a plurality of self-expanding annular segments, which are assembled in succession on a bearing shaft displaceable in the catheter.

16. The high-frequency application device as claimed in claim 1, characterized in that the HF contact elements are manufactured from a material having good X-ray contrast.
